(19) United States
(12) Patent Application Publication
(10) Pub. No.: US 2016/0213296 A1
(43) Pub. Date: Jul. 28, 2016

(54) SCREENING SYSTEM FOR FATIGUE AND STRESS

(71) Applicants: Hitachi Systems, Ltd., Tokyo (JP); FATIGUE SCIENCE LABORATORY INC., Osaka (JP)

(72) Inventors: Osamu Kikuchi, Tokyo (JP); Takayuki Matsubara, Tokyo (JP); Masutoyo Miyamoto, Tokyo (JP); Seiki Tajima, Hyogo (JP); Junichi Koizumi, Kanagawa (JP); Yosuke Kataoka, Hyogo (JP); Hirohiko Kuratsune, Osaka (JP); Yasuyoshi Watanabe, Hyogo (JP); Yoshiki Nishizawa, Osaka (JP)

(21) Appl. No.: 14/917,516

(22) PCT Filed: May 28, 2014
(86) PCT No.: PCT/JP2014/064088

§ 371 Date: Mar. 8, 2016

(30) Foreign Application Priority Data
Sep. 11, 2013 (JP) 2013-187913

(51) Int. Cl. A61B 5/16; A61B 5/04; A61B 5/00; A61B 5/024; A61B 5/0402

(52) U.S. Cl. CPC A61B 5/165; A61B 5/02405; A61B 5/0402; A61B 5/4035; A61B 5/04012

(57) ABSTRACT

The system enables a subject himself to decide on a proper remedial measure, without obtaining particular instruction directly from a medical professional, by providing an advice corresponding to a decision criteria of a plurality of classifications (3x4=12) stored in the storage unit, based on a plurality of autonomic nerve function age ranks and a plurality of autonomic nerve function age ranks

A screening system for fatigue and stress has a storage unit which, during screening for fatigue and stress, stores master data composed of reference values for each age, a decision unit which decides by comparing a measurement data obtained by electrocardiogram and pulse wave measurement of the subject with the reference value, and outputs a result of decision classified into the plurality of classifications, and a computing unit which receives the decision results, and calculates autonomic nerve function age ranks, wherein the decision means has an autonomic nerve decision unit which decides autonomic nerve strength, and an autonomic nerve balance decision means which decides the autonomic nerve balance.

### TECHNICAL FIELD

The present invention relates to a screening system for fatigue and stress.

More specifically, the present invention relates to a fatigue screening system capable of automatically measuring and analyzing a biological data (electrocardiogram and pulse wave) of a subject, and enabling the subject himself/herself to easily comprehend his/her own fatigue and stress from the analysis result, and receive evaluation corresponding to the fatigue and stress.

### BACKGROUND ART

Japanese Patent Application Laid-Open Publication No. 2010-234000 (Patent Literature 1) and No. 2001-204714 (Patent Literature 2) are examples of the background art of the present field of art.

Patent Literature 1 teaches a mental stress evaluation unit including “a biological information measuring part 5 measuring the cardiac cycle and respiratory cycle of the subject, an average cycle analysis part 9 obtaining an average cardiac cycle and an average respiratory cycle from the cardiac cycle and respiratory cycle measured in the biological information measuring part 5, a cardiac variability analysis part 10 computing cardiac cycle intervals RR (n) and RR (n+k) at the n−th beat and (n+k)th beat to an optional variable n (n is an integer) and an optional constant k (k>=1) of the cardiac cycle, and inputting them as coordinate points to two-dimensional coordinate axes, and a mental stress evaluation part 11 having a respiratory cycle variability correcting part 12 computing a ratio r of the average respiratory cycle to the average cardiac cycle to make a correction of k=r, and applying quantitative processing to a set of coordinate points after making a correction to acquire a quantitative value on the biological information of the subject and to evaluate it as a stress”, to thereby “analyze a combination of cardiac variability and respiratory information” (refer to abstract).

Further, Patent Literature 2 discloses a metal stress judging device including “a means for obtaining the heartbeat equivalent signal and the respiratory vibration equivalent signal of an examinee, a first conversion means for performing time/frequency conversion to time-axial heartbeat interval data of the heartbeat equivalent signal, a data specifying means for specifying only data in a frequency band equal to or lower than a prescribed frequency among the pieces of data converted by this first conversion means, a second conversion means for converting frequency/time to data specified by this data specifying means, a movement averaging means for performing movement averaging processing to the time-axis heartbeat interval data converted by this second conversion means based on the respiratory vibration equivalent signal, and a stress analytic means for analyzing stress based on the heartbeat interval data movement-averaged by this movement averaging means”, to thereby “provide a mental stress judging device capable of reducing errors based on the sudden change of a heart rate” (refer to abstract).

### CITATION LIST

### Patent Literature

[PTL 1] Japanese Patent Application Laid-Open Publication No. 2010-234000
[PTL 2] Japanese Patent Application Laid-Open Publication No. 2001-204714

### SUMMARY OF INVENTION

### Technical Problem

PTL1 teaches an arrangement of a mental stress evaluation unit by analyzing a combination of cardiac variability and respiratory information.

Further, PTL2 teaches an arrangement of a mental stress judging device capable of reducing errors based on the sudden change of heart rate.

However, the mental stress evaluation unit taught in PTL1 and the mental stress judging device taught in PTL2 are special devices, and there are no considerations on enabling an ordinary subject to measure stress objectively anytime and anywhere, and to enable the subject to comprehend stress in a visualized manner using a portable general-purpose mobile information terminal.

Conventionally, depression and other mental diseases are generally decided in a subjective manner through medical examination by interview or meeting with a doctor, an industrial physician, a public health nurse and the like. It was not easy for mental patients to take such examination, especially for those caring the public eye, and another drawback was that time was required for diagnosis, including the wait for examination.

Therefore, the present invention provides a screening system for fatigue and stress enabling the current fatigue and stress state to be comprehended anytime and anywhere by anyone without having to prepare a special device, and providing information helpful to take proper remedial measures.

For example, the invention provides a screening system for fatigue and stress that measures an electrocardiogram and a pulse wave at the same time, measures a state of autonomic nerve based on the electrocardiogram and pulse wave data, and numerically converting the degree of fatigue and stress tendency to enable uniform management of fatigue and analysis result data.

In further detail, the invention provides a screening system for fatigue and stress realizing a fatigue and stress screening cloud system capable of creating and outputting a fatigue level measurement result report including an autonomic nerve evaluation information corresponding to the fatigue and analysis result data.

Furthermore, the invention provides a screening system for fatigue and stress diagnosing fatigue and stress comprehensively based on strength and balance of the autonomic nerve, to enable output of evaluation according to the state of fatigue and stress of the subject, and to enable non-professionals who are not medical professionals to easily comprehend remedial measures by referring to detailed comments.

### Solution to Problem

In order to solve the above-mentioned problems, the present invention includes a unit measuring a strength and balance of autonomic nerve, and deciding an autonomic nerve function based on the measurement result

For example, a screening system for fatigue and stress according to the present invention includes, upon diagnosing fatigue and stress, a storage unit storing a reference value of each age as master data, a decision unit for comparing the measurement data obtained by measuring the electrocardiogram and pulse waves of the subject with the reference value, and outputting a decision result being categorized into a plurality of classifications, and a computing unit computing the autonomic nerve function age based on the received decision result,

the decision unit includes

an autonomic nerve decision unit deciding a strength of the autonomic nerve, and an autonomic nerve balance decision unit for deciding a balance of the autonomic nerve,

the autonomic nerve balance decision unit includes

an autonomic nerve function decision unit comparing the measurement data with a reference value showing a strength of the autonomic nerve stored in the storage unit, or a sympathetic nerve/parasympathetic nerve (LF/HF) balance reference value, deciding a plurality of autonomic nerve function age ranks N (such as three classifications: smaller than a low value, a high value or higher, and other standard values) or a plurality of sympathetic nerve/parasympathetic nerve (LF/HF) rank M (such as four classifications: low value, reference, high value and extremely high), and providing the decided balance state in a comment, and an autonomic nerve function comprehensive decision unit for providing advice respectively corresponding to the plurality of classifications (N×M=12) of the decision reference values based on the plurality of autonomic nerve function age ranks and the plurality of sympathetic nerve/parasympathetic nerve ranks, and
providing an advice regarding the plurality of decision reference values other than “warning”, “caution” and “normal”, by comparing the decision result of the autonomic nerve function comprehensive decision unit with the decision reference value.

### Advantageous Effects of Invention

Since the present invention constructs the screening system for fatigue and stress by a client-side mobile information terminal, a biological measuring instrument, and a cloud-side fatigue analysis server, the client-side subject should simply prepare a known mobile electrocardiogram and pulse wave measuring instrument and mobile information terminal, to enable anyone to comprehend a fatigue level measurement result, such as the degree of stress, by a numerical value anytime and anywhere in a short time. As a result, the subject himself/herself can easily be notified of the level of depression or other mental diseases and the remedial measures thereof, without having to be present in a medical examination with a medical professional, and without having to worry about the public eye, so that the system can be used conveniently, and can be adopted, for example, as measures for health management of employees in a company (reducing the number of long-term absentee) or health enhancement (prevention of diseases caused by fatigue and stress) of residents in a municipality.

Further, upon diagnosing fatigue and stress, an advice is provided corresponding to the decision result considering the strength and balance of the autonomic nerve, so that even those having no special knowledge can easily precisely comprehend the current state and remedial measures.

Furthermore, since advice corresponding to each decision reference of a plurality of classifications (3×3=12) stored in a storage unit is provided based on a plurality of autonomic nerve function age ranks and a plurality of sympathetic nerve/parasympathetic nerve ranks, the subject himself/herself can decide remedial measures correctly without receiving direct instructions from a medical professional.

Problems, configurations and effects other than those described above will become apparent from the following description of embodiments.

### BRIEF DESCRIPTION OF DRAWINGS

FIG. 1 is a configuration diagram illustrating an outline of a fatigue and stress system according to the present invention.

FIG. 2 is a configuration diagram of the fatigue and stress system according to the present invention.

FIG. 3 is a block diagram illustrating respective configurations of an electrocardiogram and pulse wave measuring instrument, a mobile information terminal, and a fatigue analysis server.

FIG. 4 is a view of a normal electrocardiogram, and a normal value thereof.

FIG. 5 is a view of a pulse wave and a calculation formula (P2/P1) of AI.

FIG. 6A is a view of a display screen example of a display unit of a mobile information terminal.

FIG. 6B is a view of the display screen example of the display unit of the mobile information terminal.

FIG. 6C is a view of the display screen example of the display unit of the mobile information terminal.

FIG. 7 is a view illustrating an example of cloud-side processing.

FIG. 8 is a view illustrating a content of information of an autonomic nerve evaluation (sympathetic nerve/parasympathetic nerve evaluation) value DB (master).

FIG. 9 is a view of a content of information of an autonomic nerve age reference value DB (master).

FIG. 10 is a view illustrating a content of information of a comprehensive evaluation DB (master).

FIG. 11 is a view illustrating a content of information of a history care DB.

FIG. 12 is a view of a report example of a fatigue level measurement report.

FIG. 13 is a flowchart showing a client-side and cloud-side sequence of the fatigue and stress system of the present invention, and respective processing steps of the electrocardiogram and pulse wave measuring instrument, the mobile information terminal, and the data center (including the fatigue analysis server).

### DESCRIPTION OF EMBODIMENTS

Now, a preferred embodiment of the present invention will be described with reference to the drawings. At first, we will describe a technical background related to the system of the present invention.

An autonomic nerve is a nerve that coordinates the functions of the body unrelated to his/her will, that includes a sympathetic nerve (dominant when he/she is active, under stress or nervous) and a parasympathetic nerve (dominant when he/she is resting, sleeping or relaxing).

Fatigue is generally triggered by the following five types of stresses.
(1) Mental stress caused by human relations and work
(2) Somatic stress such as excessive labor
(3) Physical stress such as ultraviolet radiation and noise
(4) Chemical stress caused by chemical substances and residual pesticide
(5) Biological stress such as virus and bacterial infection

These five types of stresses are entangled comprehensively, causing disorder of the balance between nervous system, immune system and endocrine system of the body, and causes fatigue.

The following tips are known as “self-care for preventing accumulation of fatigue”.
(A) Doing stretches before sleeping: It is effective to do stretches at nighttime when one wishes to relax and cause the parasympathetic nerve to be dominant, and it is also important to create a mentally and physically relaxing mood, such as by infusing aroma oil.
(B) Stop turning the bedroom into a living room: Stop watching TV, playing games or reading books in the bedroom, and acquire sound sleep by being aware that one should “sleep while in the bedroom”.
(C) Have worthwhileness of life: One would feel dull by spending every day life only doing work or duty, so that it is important to have hobbies that one enjoys to do on weekends, even small things such as gardening and going for a walk.
(D) Laugh: Laughing activates natural killer cells that repel viruses, and makes the body stronger against stress. One should bear in mind to always bring laughter into life, such as by watching comedy shows and rakugo (traditional Japanese comic storytelling).
(E) Lead a well-regulated life: The brain functions by the close interconnection of the immune system, the nervous system and the endocrine system. It is important to lead a well-regulated life to regulate the functions of the brain.
(F) Take a hot shower or do some exercise to start the morning: One feeling chronic fatigue tends to have a disordered autonomic nerve function. Taking a hot shower or doing some exercise after getting out of bed activates the sympathetic nerve instantaneously.
(G) Do unpleasant things in the morning, and do pleasant things after three o'clock: One should deal with unpleasant things in the morning, since doing unpleasant things in the afternoon may affect one's sleep.
(H) Take a warm bath at night and relax: Good sleep is the key to recovering from fatigue. Tension can be eased by improving the functions of the parasympathetic nerve, by taking a warm bath with a bath temperature of 38 to 40° C. or a footbath at night.

Further, fatigue and stress is known to interact and affect each other. Continuously receiving influence of fatigue and stress may lead to deterioration of activity, such as lowering of motivation, or in more serious situations, may cause physical complaints such as anorexia, or mental diseases such as depression.

Hitherto, a method for diagnosing fatigue and stress was generally based on self-evaluation using medical interview sheets or taking medial examination through interview or observation, and the diagnosis was mainly performed subjectively by the subject or by the interviewee.

However, the method for performing diagnosis and evaluation through interview has a drawback that the results may be biased by the evaluation of the subject himself/herself, that is, by the dispersion of interpretation of the questions on the medical interview sheet or by the difference between intentional answers and actual conditions that the subject is not aware of.

The drawback of evaluation by interview is that the results may be biased by the lack of skill of the interviewee, by whether there is a trusting relationship between the interviewee and object, or by the expectations to the interviewee.

In order to correct the above-mentioned problems of the conventional method for diagnosing fatigue and fatigue and stress, the present applicant has developed a system for screening fatigue and stress without depending on medical examination through interviews.

Now, the outline of such screening system for fatigue and stress will be described.

A screening system for fatigue and stress is a system for collecting electrocardiogram and pulse wave data using a biosensor, analyzing heart rate variability based on the data to measure an autonomic nerve condition, and comparing a strength and balance of autonomic nerve based on the measured data with a reference, to thereby numerically convert a degree of fatigue and stress tendency.

In further detail, at first, a user logs into a cloud environment from a dedicated terminal at a measurement site and the like, starts a measurement control program, and measures an electrocardiogram and pulse wave using a biosensor. Therefore, the system measures disorder of autonomic nerve balance.

A “high accuracy autonomic nerve measuring instrument” developed by Fatigue Science Laboratory Inc. can be used as an example of the biosensor.

The biosensor can measure the electrocardiogram and the pulse wave at the same time, so that measurement can be performed without being influenced by the subject property, for example, regardless of the difficulty of measuring pulse waves.

For example, the sensor of the measuring instrument may not be able to obtain pulse waves from a subject sensitive to cold, having poor circulation, or having a thick finger skin, but the electrocardiogram can be used to calculate an acceleration pulse wave and perform compensation.

The measurement can be performed in a short period of time, such as approximately 150 seconds, by pressing both index fingers onto a sensor.

The electrocardiogram and pulse wave data transmitted from the biosensor is received by the dedicated terminal.

The dedicated terminal transmits the received electrocardiogram and pulse wave data in an encrypted state to a cloud-side data center.

In the data center, the server decrypts the encrypted electrocardiogram and pulse wave data, refers to the data being the criteria of decision, analyzes the electrocardiogram and pulse wave data, and registers the analyzed result to a database.

The data used as decision criteria should use highly reliable criteria, such as by setting age group-based criteria based on a large number of subject data measured for example at medical checkup centers, and verifying the data with other fatigue and stress biomarkers at research institutions.

A report is created based on the analyzed result having been analyzed by a server within the data center.

The dedicated terminal is logging into the cloud server, so that it outputs (displays or prints) the analyzed result report in the cloud server.

The communication between the dedicated terminal and the data center is performed, for example, via encrypted communication, and by saving and managing health information at the secure and environment-friendly cloud-side data center, it is possible to prevent information leakage caused by intrusion of a third party.

No data such as subject data and measurement results is saved in the dedicated terminal used at the measurement site and the like, so that health information (personal information) will not be leaked even if the dedicated terminal is stolen or lost.

The above arrangement enables to retain secret health information of the measurer reliably and safely at the cloud side.

Furthermore, the health information stored in the cloud-side data center is stored and managed in a centralized manner, and the accumulated data can be utilized for many purposes, such as analysis of data for each individual or for each group.

Further, the measured result is output as a result report so that it can be conveyed in an intelligible manner.

The report includes, from top to bottom, a basic information and measured information area, an autonomic nerve function age area, a heart rate variability area, a sympathetic/parasympathetic nerve area, and an autonomic nerve evaluation area.

The basic information and measured information area displays information related to basic information regarding the measurer (such as name, sexuality and age), and measurement information (such as measurement date and measurement time).

The autonomic nerve function age area displays the result of comparison of the autonomic nerve function age at the time of measurement and the age of the measurer in a graph showing the degree of fatigue.

In the graph, a vertical axis represents an autonomic nerve function (CCVTP), and a horizontal axis represents age.

The autonomic nerve function represents the strength of the autonomic nerve (sympathetic nerve and parasympathetic nerve), and a green line b indicates an age average that declines with age. A face mark represents, as a function age, which age average the strength of the autonomic nerve according to the current measurement value corresponds to. As the mark moves upward, it shows higher autonomic nerve functions, and tendency of fatigue and the like causes the mark to move downward. A red line c represents a low value of the reference value of that age. It can be used as an index indicating that the function is deteriorated if the value falls below the red line. A blue line a represents a high value of the reference value of that age. If the value is higher than the blue line, measurement noise is suspected, so that measurement must be performed again. If the re-measured value is the same result, the value is considered correct, and it can be determined that the autonomic nerve function is extremely high.

The heart rate variability area displays an average heart rate during measurement and heart rate variability (variability of heart beat intervals) condition (fluctuation of heart rate variability). The part where the waveform drops to the bottom represents measurement failure (data deficiency).

The sympathetic/parasympathetic nerve area displays the balance between sympathetic nerve and parasympathetic nerve by graphs and numerical values.

The autonomic nerve is composed of the sympathetic nerve and the parasympathetic nerve, and the balance between these nerves is illustrated in this area. The face mark displayed toward the right indicates that the sympathetic nerve is dominant (that the subject is nervous or under stress), and the face mark displayed toward the left indicates that the parasympathetic nerve is dominant (that the subject is relaxed). As for the sympathetic nerve and the parasympathetic nerve, it is an ideal state to have the sympathetic nerve dominant during an active period and the parasympathetic nerve dominant during a resting period.

The autonomic nerve evaluation area displays the autonomic nerve state comprehensively in a three-grade evaluation, and displays the current state and an advice on how to realize a better state.

The result of decision of the state of the autonomic nerve at the time of measurement based on a comprehensive evaluation of function (strength) and balance is displayed, and the description on the autonomic nerve state at the time of measurement and an advice for improving the autonomic nerve function (strength) and balance are displayed.

According to the above-described screening system for fatigue and stress, it becomes possible to find a high-risk subject at an early stage and to cope with the subject, by analyzing periodic individual data through sampling during health checkups. It becomes possible to find a high-risk subject at an early stage and to cope with the subject, by analyzing periodic individual data through sampling during health checkups.

The numerical value data related to autonomic nerve and the numerical value data of the degree of fatigue and stress tendency registered in the database can be used to evaluate the effects and efficacy of products and services as numerical value.

Numerically converting the fatigue level and stress tendency and comparing the same with reference values enables to eliminate bias, and the management of history of numerical value data enables to comprehend the tendency of the state of fatigue and stress. By using the system together with medical examination through interview, screening having a higher accuracy is made possible.

Further, numerically converting the degree of fatigue and stress tendency based on the autonomic nerve enables easier understanding even for non-health professionals.

However, analyzing is performed based on only instantaneous measurement, that is, electrocardiogram and pulse waves measured for only approximately 150 seconds within a day, and depending on the measurement condition, that is, depending on the measurement timing, erroneous screening result may be derived. In other words, the system was not exactly an appropriate screening method, and had some drawbacks from the viewpoint of providing accurate screening. Some results may cause unnecessary worries and anxiety of the subject.

In other words, there was no consideration on providing decision and care corresponding to everyday life.

The present invention provides a system of saving reference value based on subject measurement data acquired by a life-log (active mass analysis) system measuring the active mass for a whole day (time-series information of a day, such as in the morning, at noon, at night, before meals, after meals, before exercise, after exercise, and so on) as master, referring to the reference value when deciding the screening decision of fatigue and stress based on the subject measurement data, and deciding the result comprehensively, so that a report information for enabling the subject to perform care corresponding to lifestyle can be provided, and a system capable of effectively utilizing the present system can be provided.

### Embodiment 1

The present embodiment illustrates an example of a system using a portable biosensor to objectively decide the fatigue state on the spot, and outputting the result as report using a mobile information terminal (also referred to as client terminal).

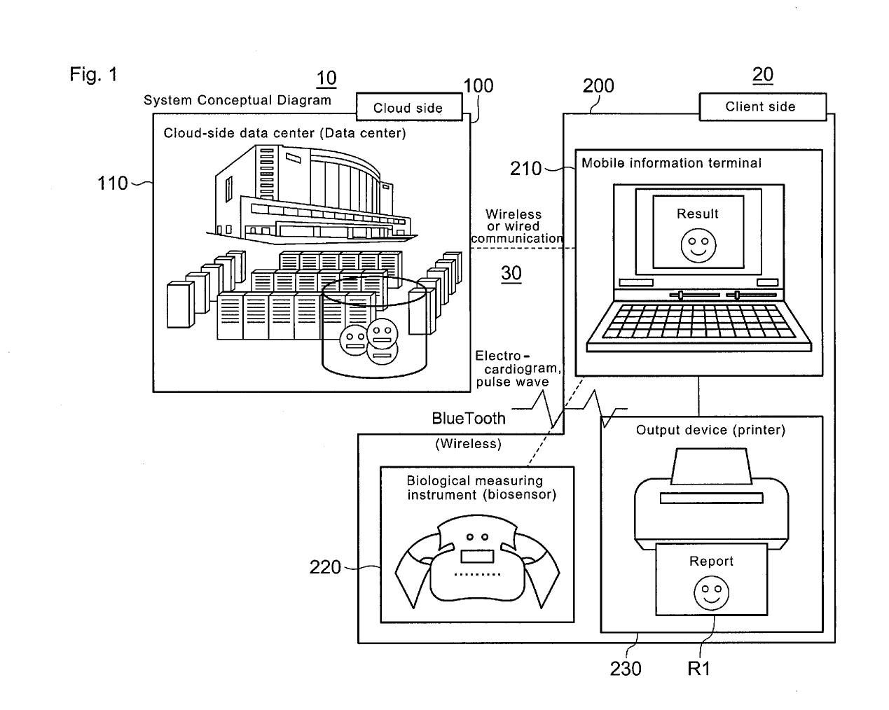

FIG. 1 is a view of an outline of a screening system for fatigue and stress.

In the drawing, a screening system for fatigue and stress includes a cloud-side device 10, a client-side device 20, and a (wired/wireless) network 30.

The cloud-side device 10 includes a data center 110 having a database (DB). The data center 110 of the cloud-side device 10 includes a control and analysis program (not shown) having a function for controlling biological data measurement and analysis, and also performing retrieval processing of measurement data. It has a function to receive measured data transmitted from the client-side device 20, analyze the data, create an analysis result report based on the analyzed result, and transmit the result to a mobile information terminal 210 at the client-side device 20.

Now, the use of the data center 110 realizes a secure image security, and the user does not have to worry about maintenance and operation of the server for analyzing measurement data. The group of programs such as the control and analysis program and database (DB) are stored in the server. The client can log into the virtual environment, and use the programs in the server.

The control and analysis program can be a program for controlling the measurement processing, a program for analyzing the information of the biological measuring instrument, a program performing retrieval processing of the measurement history, and a program for generating a report of the result of fatigue level measurement, and these programs are stored in the database.

The client-side device 20 includes a mobile information terminal 210, a biological measuring instrument (biosensor) 220, a printer device (output device) 230, and so on. The mobile information terminal 210 of the client-side device 20 sends and receives data between the biological measuring instrument 220 and the cloud-side device 10.

In other words, it receives the measurement data of the subject measured by the biological measuring instrument 220, performs determined processes, and transmits the data together with basic information of the subject to the data center 110 side.

Further, it has a function to receive an analysis result report (described later) R1 transmitted from the cloud-side device 10, and print and output the report using a printer 230 as the output device.

It is desirable to consider portability and to perform wireless communication (4G circuit: LTE being the standard) between the cloud-side and the client-side. However, when an intracompany network is used within a company, wired network can be used.

The analysis result report includes “basic information”, “autonomic nerve function age”, “heart rate variability”, “sympathetic/parasympathetic nerve”, and “autonomic nerve evaluation”. The detailed contents of the report will be described later.

The biological measuring instrument 220 has a function to measure electrocardiogram and pulse wave at the same time, and transmit the result to the mobile information terminal 210.

The printer 230 is used to print out the report, and it should preferably be a color printer, since “warning”, “caution” and “normal” are shown in the printed report by “red”, “yellow” and “blue” face marks, for example.

The screening system for fatigue and stress performs communication with the biological measuring instrument, and the server should have as much function as possible, other than the function for transmitting biological data to the cloud-side server, and various data should also be integrally managed at the server side. Thereby, a configuration considering extensibility and security is realized. Hereafter, an example of the configuration will be described.

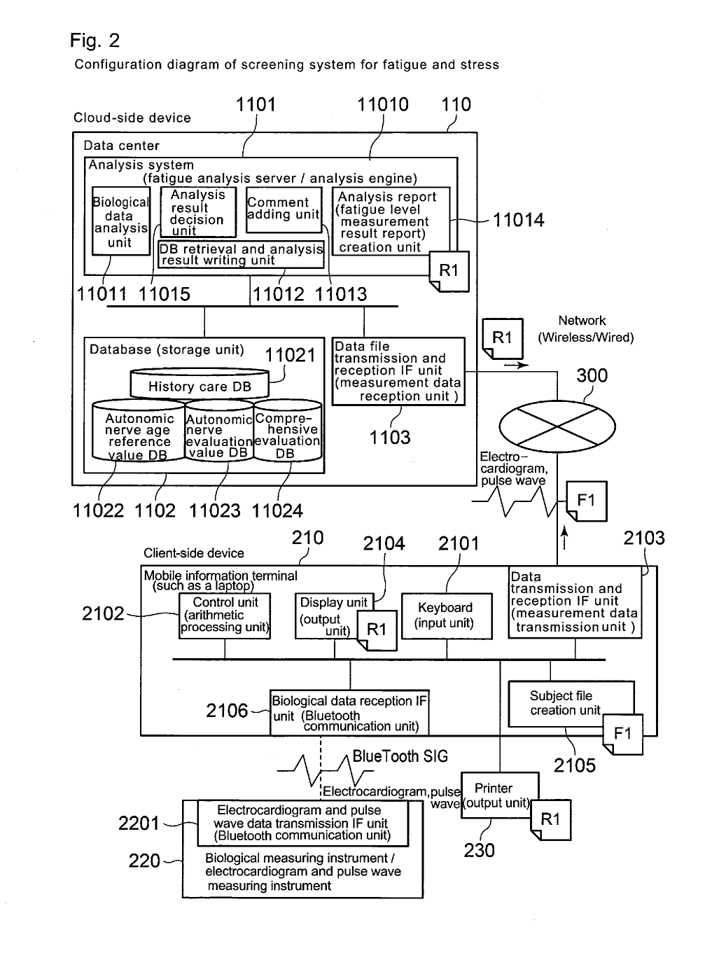

FIG. 2 is a configuration diagram of the screening system for fatigue and stress according to the present invention.

In the drawing, the data center 110 includes an analysis system. The analysis system includes a fatigue analysis server 1101, a database (storage unit) 1102, a data file transmission and reception IF unit 1103, and so on.

The fatigue analysis server 1101 includes an analysis engine 11010 that operates based on a control and analysis program not shown.

The analysis engine 11010 includes a biological data analysis unit 11011, a DB retrieval and analysis result writing unit 11012, a comment adding unit 11013, an analysis report (fatigue level measurement result report) creation unit 11014, an analysis result decision unit 11015, and so on.

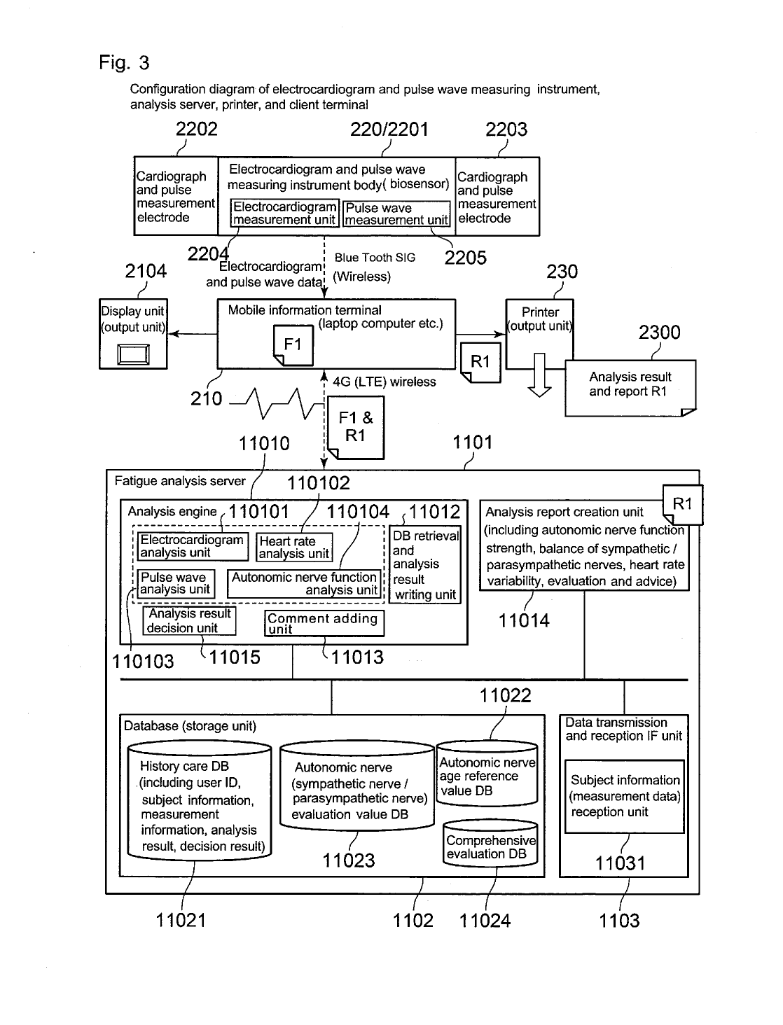

The biological data analysis unit 11011 has a function to receive and analyze the electrocardiogram and pulse wave data transmitted from the mobile information terminal 210 of the client-side device, and output CCVTP, LH and HF as fatigue level decision values. The detailed configuration and functions are described with reference to FIG. 3.

The DB retrieval and analysis result writing unit 11012 has a function to search the database 1102, extract necessary information, and store the analysis result in the database 1102.

The analysis result decision unit 11015 has a function to decide the analysis result by the biological data analysis unit 11011. The details will be described later.

The analysis report (fatigue level measurement result report) creation unit 11014 creates a report including the analysis result, evaluation and comments, and uses the report as a fatigue level measurement result report R1.

The comment adding unit 11013 has a function to add the analysis report R1 to the evaluation and comments (stored in the database 1102) according to the result of decision of the analysis result decision unit 11015 deciding the analysis result by the biological data analysis unit 11011.

The database (storage unit) 1102 has a history care DB 11021. Further, the database includes a master DB used for deciding the measurement result, in other words, an autonomic nerve age reference value DB 11022, an autonomic nerve (sympathetic nerve/parasympathetic nerve) evaluation value DB 11023, a comprehensive evaluation DB 11024, and so on.

The history care DB 11021 includes subject information (basic information) and stores the analysis result having analyzed the biological data and the result of decision as history care information.

The autonomic nerve age reference value (master) DB 11022 stores an “autonomic nerve function analysis age reference value” (average value per age: such as low value, reference value, and high value of each age) used for deciding the measurement result.

The autonomic nerve (sympathetic nerve/parasympathetic nerve) evaluation value DB 11023 stores the “sympathetic/parasympathetic nerve reference value” (evaluation: comment of state for each of the four classifications, and standard values (low value, reference value, and high value) of each of the four classifications) used for deciding the measurement result.

The comprehensive evaluation DB 11024 stores a “comprehensive evaluation reference value” used for deciding the measurement result.

The details of the detailed information of each DB will be described later.

The data transmission and reception IF unit (fatigue level measurement result report and subject information transmission and reception unit) 1103 monitors biological information from the client. The unit has a function to receive subject information F1 of the mobile information terminal 210 of the cloud-side device 10, and transmit the fatigue level measurement result report to the mobile information terminal 210 of the client-side device 20. The subject information F1 and the fatigue level measurement result report R1 should be transmitted and received in file formats.

The mobile information terminal 210 can be, for example, a laptop. In the present embodiment, it is illustrated as a dedicated terminal having a file creating function for each subject (user/client).

The mobile information terminal 210 includes a keyboard (input unit) 2101, a control unit (arithmetic processing unit) 2102, a data transmission and reception IF unit 2103 (subject information and fatigue level measurement result report transmission and reception unit), a display unit (output unit) 2104, a subject file creation unit 2105, a biological data reception IF unit (Bluetooth (Registered Trademark) communication unit) 2106, and so on. Communication by the biological data reception IF unit 2106 utilizes a well-known Bluetooth.

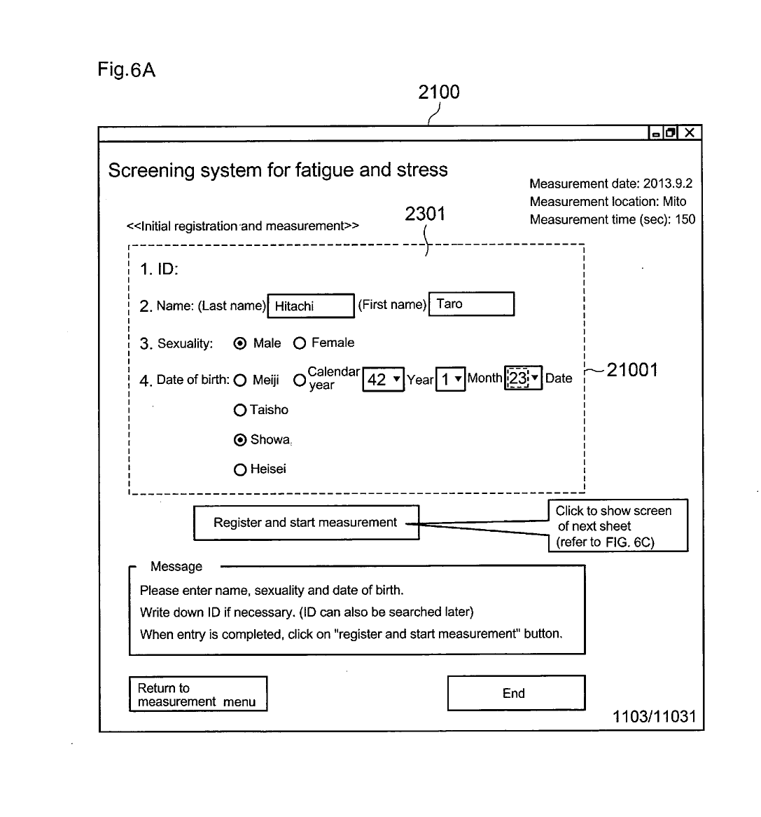

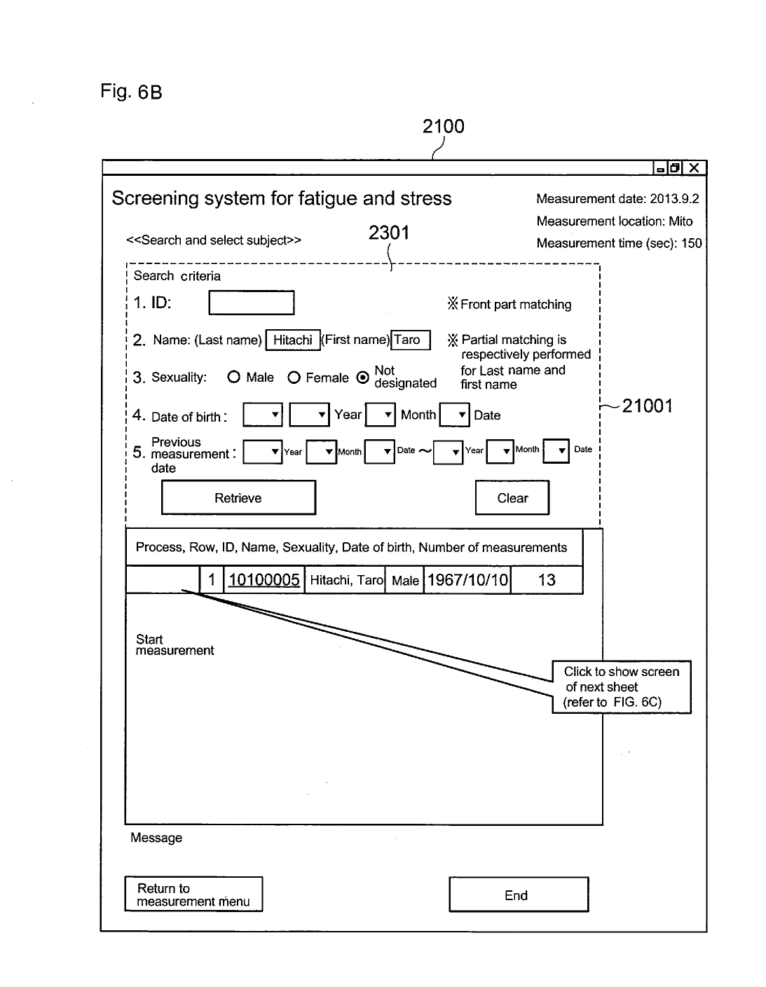

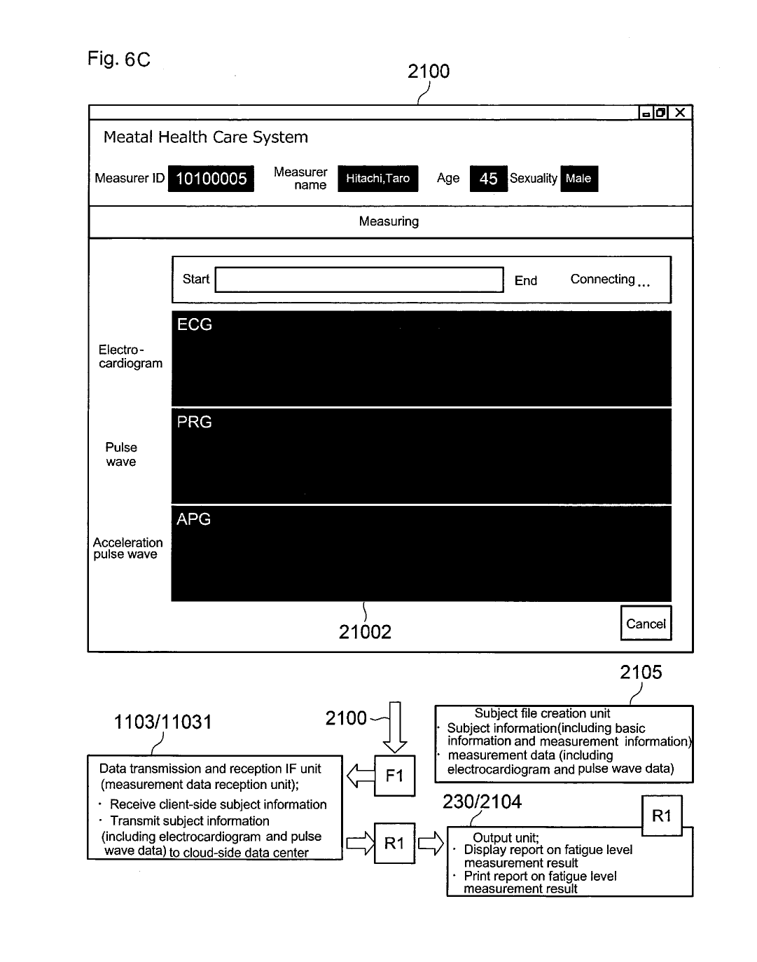

The keyboard (input unit) 2101 is used for entering the basic information and measurement information of the subject (measurer/client) guided by various items on a subject information input screen (refer to FIG. 6) based on subject information entries on a display controlled by the control unit 2102.

The control unit (arithmetic processing unit) 2102 has a function to control the various units. For example, it logs into the server in a virtual environment from the client side to the cloud side, and activates a measurement processing program on the cloud side. Then, based on the program, it performs measurement guidance on the display unit 2104 of the biological measuring instrument 220, and controls the processing of measurement data in the biological measuring instrument 220. That is, signup of a subject, measurement location and measurement time are registered according to a guidance display on the display unit 2104, and control is performed to process measurement data of the biological measuring instrument 220. Further, control is performed to retrieve past measurement results.

The data transmission and reception IF unit (subject information and fatigue level measurement result report transmission and reception unit) 2103 has a function to transmit the subject information F1 to the fatigue analysis server 1101 side, and receive the fatigue level measurement result report R1 transmitted from the fatigue analysis server 1101.

The display unit (output unit) 2104 has a function to display a guidance to prompt the entry of subject information, and display the entered basic information of the subject, the measurement data of the biological measuring instrument 220 (electrocardiogram and pulse wave), the fatigue level measurement result report R1, and so on.

Upon receiving the basic information entered through a keyboard 2101, the subject file creation unit 2105 has a function to create a desired subject file (CSV file) F1 as subject information based on a file creation application (not shown).

The biological measuring instrument 220 is composed of a electrocardiogram and pulse wave measuring instrument, and has a transmission IF unit (Bluetooth communication unit) 2201 for measuring electrocardiogram and pulse wave data at the same time, and transmitting and receiving data with a cloud side.

The electrocardiogram and pulse wave data transmission IF unit 2201 uses Bluetooth for communication.

A printer (output unit) 230 is for printing the fatigue level measurement result report R1. The subject can visually comprehend the result of fatigue level measurement based on the print printed by the printer 230 or the display on the display unit (output unit) 2104. The report should be printed in color, so as to represent “warning”, “caution” and “normal” using different colors, such as “red”, “yellow” and “blue” face marks.

A network 300 is for transmitting and receiving information between the data center 110 and the mobile information terminal 210, and it can either be wireless or wired, whereas the present embodiment utilizes a wireless 4G (LTE) network.

FIG. 3 is a configuration diagram illustrating an example of an electrocardiogram and pulse wave measuring instrument, an analysis server, a printer, and a client terminal.

In the drawing, the biological measuring instrument 220 is composed of a known electrocardiogram and pulse wave measuring instrument, and includes an electrocardiogram and pulse wave measuring instrument body (biosensor) 2201. Electrocardiogram and pulse measurement electrodes 2202 and 2203 on which the subject contacts his/her finger tips are provided on both ends of the electrocardiogram and pulse wave measuring instrument body (biosensor) 2201.

An electrocardiogram measurement unit 2204 for measuring the electrocardiogram and a pulse wave measurement unit 2205 for measuring the pulse wave using current flown from finger to finger in contact with the electrodes 2202 and 2203 are provided within the electrocardiogram and pulse wave measuring instrument body (biosensor) 2201.

Electrocardiogram waves and pulse waves are measured simultaneously using the electrocardiogram and pulse wave measuring instrument body (biosensor) 2201. the measurement time can be, for example, one minute at minimum, and the measured value is transmitted to the mobile information terminal 210 in real time.

The fatigue analysis server 1101 includes the analysis engine 11010 and the DB retrieval and analysis result writing unit 11012. The analysis engine 11010 includes an electrocardiogram analysis unit 110101, a heart rate analysis unit 110102, a pulse wave analysis unit 110103, an autonomic nerve function analysis unit 110104, and so on.

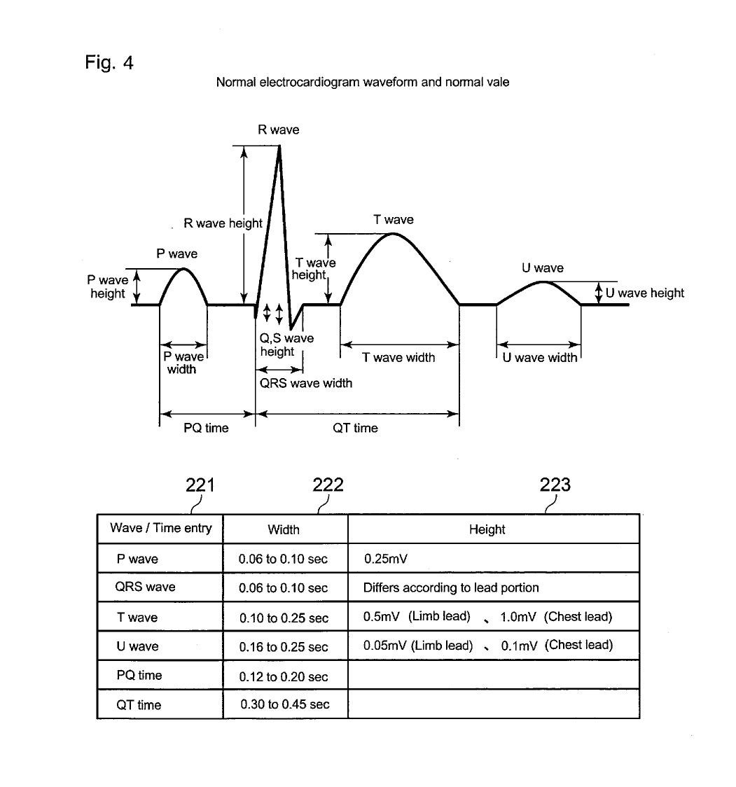

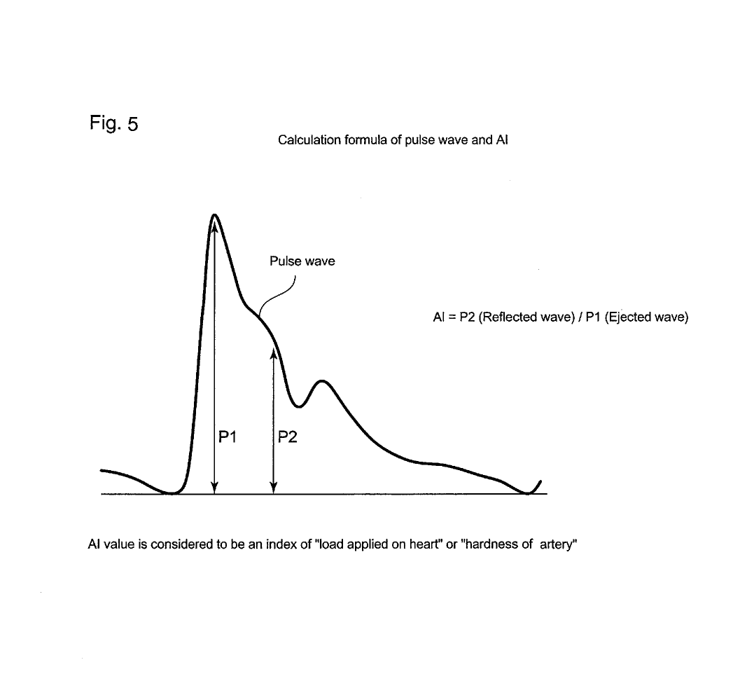

The electrocardiogram analysis unit 110101 analyzes the electrocardiogram (refer to FIG. 4) of the subject, the pulse wave analysis unit 110103 analyzes the pulse wave (FIG. 5), and the heart rate analysis unit 110102 analyzes the heart rate variability (differences of cardiac cycle) based on the electrocardiogram and pulse waves.

That is, the electrocardiogram analysis unit 110101 analyzes the waveform including P wave, R wave, T wave, QRS wave and the like as measured electrocardiogram data.

Based on the analysis, if the waveform of the electrocardiogram data does not have a P wave, or the R wave does not have equal intervals, it can be comprehended that a left ventricular hypertrophy is suspected if there is an irregular pulse R wave, that a myocardinal ischemia or angina attack is suspected if the ST portion is dropped horizontally, or that a high potassium crystal or myocardinal infarction is suspected if the T wave is pointed, for example.

Further, the pulse wave analysis unit 110103 analyzes an “AI value” calculated by a ratio of “ejected wave P1” that occurs by the heart being contracted to send blood throughout the whole body and “reflected wave P2” that occurs by the ejected wave being reflected on peripheral artery and artery branches when flowing throughout the whole body (“P2/P1”).

The autonomic nerve function analysis unit 110104 measures the state of the autonomic nerve from the electrocardiogram and pulse waves, and analyzes fatigue and stress. That is, the autonomic nerve function analysis unit 110104 analyzes the balance and strength of autonomic nerves that cannot be controlled by one's will based on the electrocardiogram and pulse waves, analyzes the heart rate variability based on the autonomic nerve, and performs analysis to comprehend the state of stress based on these analysis results in numerical values.

The autonomic nerve includes a sympathetic nerve that is dominant when he/she is exercising to bring the body to an excited state, during which adrenaline and noradrenaline are active, and a parasympathetic nerve that is dominant when the body is relaxing, such as during meals or sleeping, during which acetylcholine are active.

Therefore, it is possible to analyze the strength of functions and balance of the sympathetic nerve and the parasympathetic nerve.

The state of the autonomic nerve is obtained from the heart rate variability (short or long). The degree of fatigue analyzes the strength of the autonomic nerve, and performs analysis by comparing the strength of the autonomic nerve with a reference (reference value of the evaluation reference DB). The stress tendency is analyzed and decided by analyzing the state of balance of the autonomic nerve (sympathetic nerve and parasympathetic nerve), compares the balance of the autonomic nerve with a reference (various information including the sympathetic nerve/parasympathetic nerve evaluation value DB, the autonomic nerve age reference value DB, and comprehensive evaluation DB).

The heart rate analysis unit 110102 analyzes the heart rate variability. The heart rate variability measures the variability of each heart beat as an index of tension of the autonomic nerve of the heart. The heart rate variability is reduced by age, and especially in aged people, deformation of the cardiovascular system is accelerated. Then, the unit evaluates all heart rate variability evaluation, and analyzes a power spectrum of frequency component of cardiac cycle variability.

It is well known that the autonomic nerve function is varied by mental stress and the like, but by using heart rate variability, such as when mental stress is applied, high frequency component (HF component: 0.20 to 0.35 Hz/reflecting change of respiration) is suppressed and intermediate frequency component (LF component: 0.05 to 0.20 Hz)/reflection of pressure receptor system) is increased.

Therefore, it is possible to use the heart rate variability and indicate the current state, the change, the evaluation and remedial advice on the autonomic nerve function in an objectively visible manner.

The DB retrieval and analysis result writing unit 11012 has a function to receive the analysis result of the respective analysis units, retrieve the master DBs 11022, 11023 and 11024 of the database 1102, and extract necessary information. Further, it has a function to write the analysis result and the decision result to the history care DB 11021 of the database 1102.

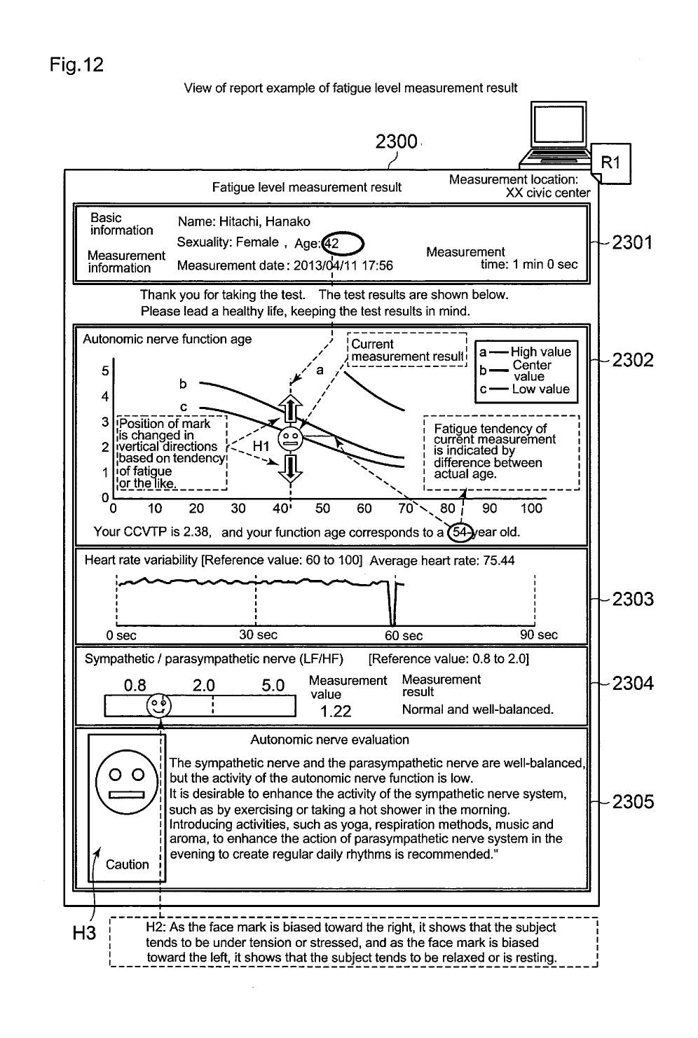

An analysis report creation unit 11014 has a function to create an analysis report (refer to FIG. 12) including information such as the autonomic nerve function strength, the balance of sympathetic/parasympathetic nerves, the heart rate variability, the evaluation and advice, based on the analysis result.

The data transmission and reception IF unit 1103 includes a subject information and biological data (subject file) reception unit 11031.

The subject information and biological data (subject file) reception unit 11031 receives subject information and biological data from the mobile information terminal 210.

The mobile information terminal outputs a fatigue level measurement result report in the cloud environment to which the terminal is logged in.

The transmission and reception of CSV files including biological data (measureed data) and report should preferably be performed via secure communication (encrypted communication) considering theft or loss.

FIG. 4 is a view of a normal electrocardiogram diagram, and a normal view thereof.

In the drawing, the electrocardiogram waveform includes P wave, R wave, T wave and U wave, and shows the heights and wave widths of the waves.

FIG. 5 is a view illustrating a pulse wave and a calculation formula (P2/P1) of AI.

In the drawing, the AI value (index indicating the load applied on the heart or the hardness of the pulse wave) can be calculated based on the ratio (P1/P2) of ejection wave (P1) and reflected wave (P2) of the pulse wave.

FIG. 6 (FIGS. 6A through 6C) are views showing an example of the subject information input screen on the display screen of the display portion of the mobile information terminal.

FIG. 6A illustrates an example of registering subject information when performing measurement for the first time using the present system.

In the drawing, a display screen 2301 is displayed on the display unit 2104 of the mobile information terminal 210 to enter basic information and measurement information. In the display screen, basic information is entered through the input unit (keyboard) 2101 of the mobile information terminal in response to a message, and the information is registered by clicking on the “register and start measurement” (measurement start button).

Basic information includes, for example, ID, name, sexuality, and date of birth. The basic information is displayed on a display area 21001. Further, measurement date, measurement location, measurement time (seconds), and message (guidance) related to the entry operation are displayed.

FIG. 6B is an example of a screen of a subject whose past measurement history exists in the history care DB 11021 (second measurement or more), and in that case, the user retrieves the subject information, and clicks on (presses) the “start measurement” (measurement start button) on the display tag. The electrocardiogram and pulse wave measurements using the biosensor 220 is started by this operation. FIG. 6C is a drawing illustrating a frame format of an example of a screen on which measurement data is displayed when the user operates the “start measurement” (measurement start button) of FIG. 6B and starts measurement, a creation of a file for the subject based on the data, and output of a report of the result of fatigue level measurement.

In the drawing, when the measurement start button is operated and measurement is started, the system receives data of the electrocardiogram and pulse waves of the biosensor 220, and displays in real time the waveform of the electrocardiogram, the pulse wave and the acceleration pulse wave on a measurement screen display area 21002 of the display unit 2104 of the mobile information terminal 210.

The subject information (including the basic information and measured information) and the measurement data (including the electrocardiogram and pulse data) are formed as a file in the subject file creation unit 2105, and transmitted as subject file (including electrocardiogram and pulse wave data) F1 to the cloud-side data center 110 at a data file transmission and reception IF unit (subject file and fatigue level measurement result report transmission and reception unit) 1103.

That is, the client side performs the following processes.

A guidance (not shown) showing how to use the biological measuring instrument 220 and perform measurement is displayed on the display screen 2100 so that a subject performing measurement for the first time can manipulate the instrument. Further, during measurement, it displays electrocardiogram and pulse waves on the spot (refer to FIG. 6C), and performs operation to enable confirmation that correct measurement is performed. The details are as follows.

### <Receive Measurement Location and Measurement Time (Screen)>

When starting measurement, a measurement information indicating the “measurement location” and the like registered in the history care DB 11021 is selected. When the “measurement location” is new, information related to the “measurement location” entered through a keyboard is received, and the location is additionally registered in the history care DB 11021. A “measurement time” is displayed as default on the display screen, and it is changed only when needed.

### <Select Subject (Screen)>

When the subject is registered, the past measurement history is searched to specify the subject, and the measurement is started.

### <Register New Subject (Screen)>

In the case of a new subject, name, sexuality and birth date are received and registered in the history care DB 11021, before starting measurement.

### <Start Measurement Processing at Mobile Information Terminal Side, and Monitor (Internal Processing)>

Measurement processing on the mobile information terminal side is started, the measurement processing by the biological measuring instrument 220 is monitored, and reception of the measurement result from the biological measuring instrument is waited.

<Display Guidance (Screen)>

Operation procedures such as how to switch the power on the biological measuring instrument 220 is displayed on the display screen, and starting of measurement is accepted.

### <Measurement Processing at Biological Measuring Instrument (Screen)>

The electrocardiogram and pulse wave data (waveform of measurement condition) of the biological measuring instrument 220 is displayed in real time on a display area 21002. In the case of a subject whose pulse is difficult to measure, the acceleration pulse wave is calculated from the electrocardiogram, and the waveform is displayed in a similar manner. The measurement time is executed for a period of time entered as the measurement information.

<Measure and Transmit Biological Information (Internal Processing)>

The electrocardiogram and pulse wave data measured by the biological measuring instrument 220 is transmitted to the client-side mobile information terminal 210.

### <Transmit Measurement Result (Internal Processing)>

When the designated measurement time is ended, the mobile information terminal 210 displays guidance that measurement is ended on the display screen. Further, it creates a file based on the measurement data, and transmits the file to the cloud-side fatigue analysis server 1101. After transmitting the file, the file is deleted from the mobile information terminal 210 from viewpoint of security.

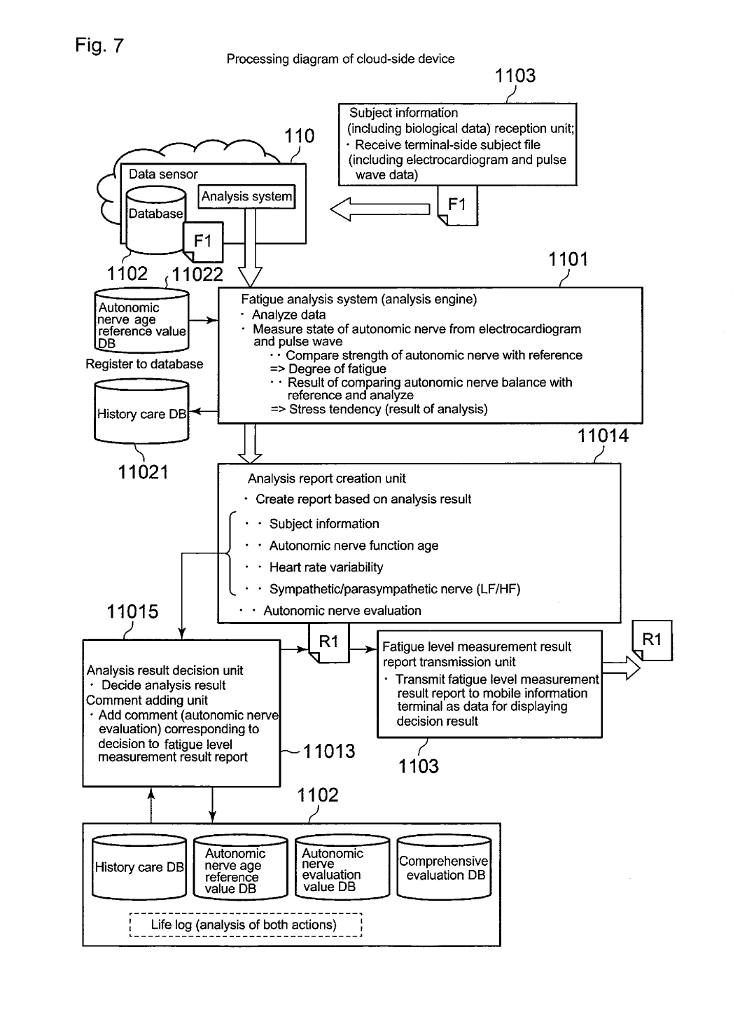

FIG. 7 is a diagram illustrating in frame format the processing flow of the cloud-side device.

In the drawing, the data center 110 receives the subject file (including the electrocardiogram and pulse wave data) from the mobile information terminal of the client-side device via a subject file (subject information and biological data) reception unit 1103. Then, the file is registered in the database 1102.

The data center 110 analyzes the electrocardiogram and pulse wave data of the subject file by a fatigue analysis system (analysis engine) 1101. Regarding data analysis, at first, the condition of the autonomic nerve is measured from the electrocardiogram and pulse wave, and thereafter, the strength of the autonomic nerve is compared with a reference information DB 11022, and the degree of fatigue is analyzed.

The analysis result of the balance of the autonomic nerve compared with the reference of the reference information DB 11022 is used to analyze the stress tendency. Then, an analysis report is created based on the analysis result in the analysis report creation unit 11014. The analysis report includes information such as subject information, autonomic nerve function age, heart rate variability, sympathetic/parasympathetic nerve (LF/HF), and autonomic nerve evaluation.

In creating the analysis report, an autonomic nerve evaluation information (autonomic nerve evaluation comment) in the autonomic nerve evaluation information DB 11023 is associated with the analysis result in an analysis result and autonomic nerve evaluation association unit 1013, and set as a fatigue level measurement result report.

That is, the following processes are performed on the cloud side.

### <Analysis Processing (Internal Processing)>

After confirming that the monitored measurement file has been received, the file is entered in the analysis engine of the fatigue analysis server 1101, and necessary index (CCVPT, LH, HF and so on) for deciding the fatigue level is calculated.

### <Store Analysis Result in DB (Internal Processing)>

The above-acquired analysis result is compared with the reference value master of the autonomic nerve age reference value DB, the autonomic nerve evaluation value DB and the comprehensive evaluation DB in the database 1102, and the degree of fatigue is decided. These values are all stored in the DB.

### <Generate and Transmit Report (Internal Processing)>

A fatigue level measurement result report is generated based on the decided degree of fatigue, and transmitted to the client-side mobile information terminal 210.

### <Receive and Display Report (Internal Processing)>

The mobile information terminal 210 receives the fatigue level measurement result report from the fatigue analysis server 1101, and displays the report.

### <Execute Printing of Report (Screen)>

Further, execution of printing of report is accepted.

### <Print Report (Printed Matter)>

Then, the fatigue level measurement result is printed using the printer 230.

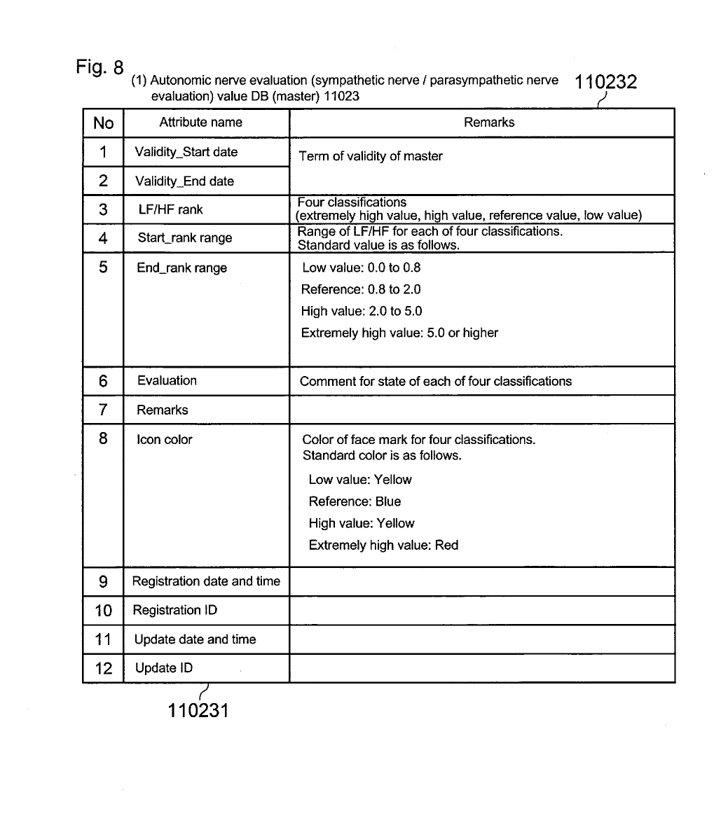

FIGS. 8 through 11 are views illustrating the contents of tables of the sympathetic nerve/parasympathetic nerve evaluation value, the autonomic nerve age reference value, the comprehensive evaluation and the history DB as typical master used in the screening system for fatigue and stress.

FIG. 8 is a view illustrating information of the autonomic nerve evaluation value, that is, the information of the sympathetic nerve/parasympathetic nerve evaluation value (master).

In the drawing, the sympathetic nerve/parasympathetic nerve evaluation value (master) includes an attribute name column and a remarks column. An attribute name column 110231 stores information such as “start date and end date of term of validity” (term of validity of master), “LF/HF rank” (four classifications: extremely high value, high value, reference value, low value), “start and end of rank range” (range of LF/HF for each of the four classifications: “standard: low value: 0.0 to 0.8”, “reference: 0.8 to 2.0”, “high value: 2.0 to 5.0, extremely high value: 5.0 and higher)”, “evaluation” (comments regarding state for each of the four classifications), “remarks”, “icon color” (color of face mark for each of the four classifications. Low value: yellow, reference: blue, high value: yellow, extremely high value: red), “registration date and time”, “registration ID”, “update date and time”, and “update ID”.

A comment can be, for example, “the sympathetic nerve is high; the parasympathetic nerve system is well balanced, but the activity of the autonomic nerve function is low. It is desirable to enhance the function of the sympathetic nerve system, such as by exercising or taking a hot shower in the morning. Introducing activities, such as yoga, respiration methods, music and aroma, to enhance the function of the parasympathetic nerve system in the evening to create regular daily rhythms is recommended.”

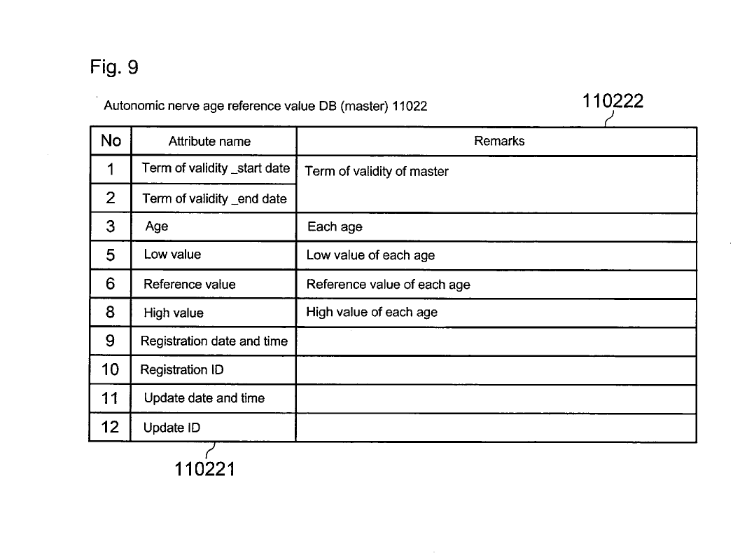

FIG. 9 is a view of an information related to an autonomic nerve age reference value (master).

In the drawing, the autonomic nerve age reference value (master) includes an attribute name column and a remarks column. The attribute name column 110211 stores information, such as “start date and end date of term of validity” (term of validity of master), “age” (each age), “low value” (low value of each age), “reference value” (reference value of each age), “high value” (high value of each age), “registration date and time”, “registration ID”, “update date and time”, and “update ID”.

The reference value formulates age-based references based on clinical trial data from a medical examination center, for example, and verification of correlation with other biomarkers from research institutions is carried out to create a highly reliable reference for deciding fatigue and stress.

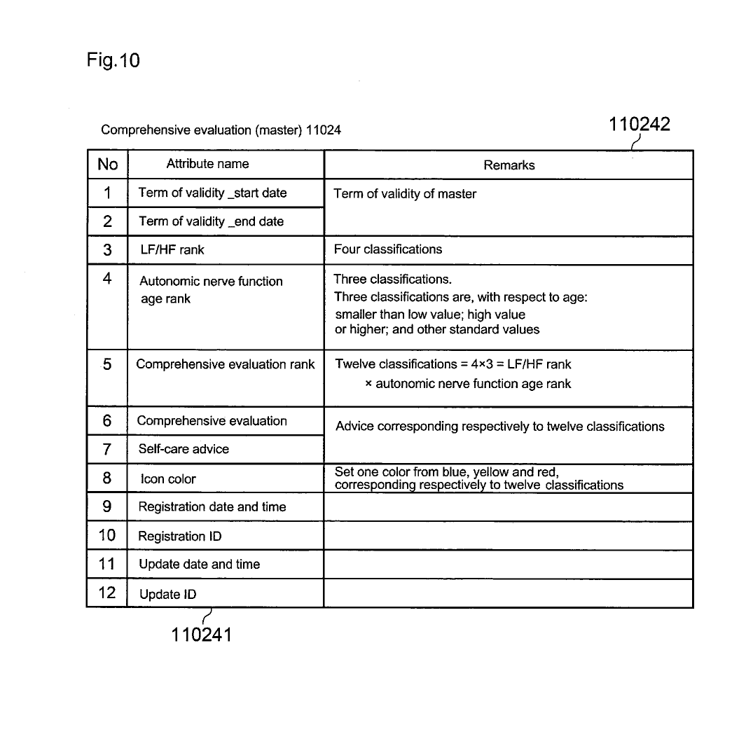

FIG. 10 is a view of an information of a comprehensive evaluation (master).

In the drawing, the autonomic nerve age reference value (master) includes an attribute name column and a remarks column. An attribute name column 110241 stores information such as “term of validity_start date”, “term of validity_end date”, (term of validity of master shown in remarks column), “LF/HF rank” (four classifications), “autonomic nerve function age rank (three classifications: a value smaller than a low value, a high value or higher, and other values which are a standard values), “comprehensive evaluation rank (ₐdᵥice corresponding to twelve classifications), “comprehensive evaluation”, “self-care advice” (advice according to the twelve classifications), icon color (single color selected from blue, yellow and red according to the twelve classifications), “registration date and time”, “registration ID”, “update date and time”, and “update ID”.

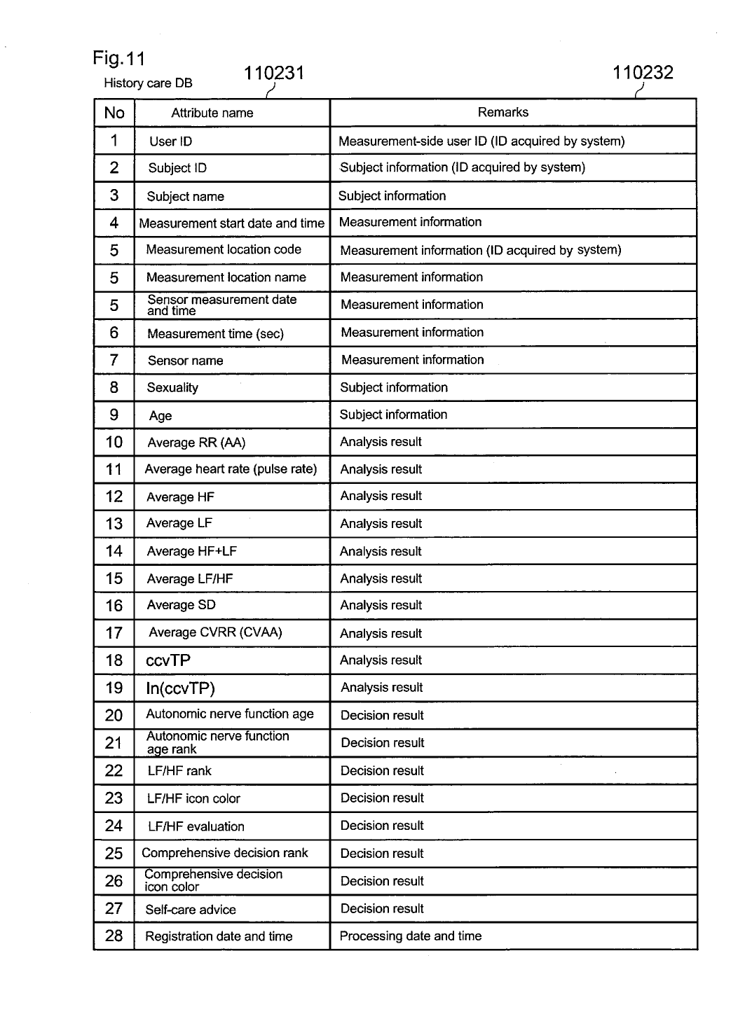

FIG. 11 is a view of an information of a history care DB.

In the drawing, the history care DB includes an attribute name column and a remarks column. The attribute name column 110231 stores information such as “user ID” (measurement-side user ID), “subject ID” (subject information), “subject name” (subject information), “measurement start date and time” (measurement information), “measurement location code” (measurement information), “measurement location name” (measurement information), “sensor measurement date and time” (measurement information), “measurement time (seconds)” (measurement information), “sensor name” (ₘₑₐₛᵤᵣₑₘₑₙt information), “sexuality” (measurement information), “age” (ₘₑₐₛᵤᵣₑₘₑₙt information), “average RR (AA)” (measurement information), “average heart rate (number of pulse)” (analysis result), “average heart rate”, (analysis result), “average HF” (analysis result), “average LF” (analysis result), “average HF+LF” (analysis result), “average LF/HF” (analysis result), “average SD” (analysis result), “average CVRR (CVAA)” ((analysis result), “ccvTP” (analysis result), “In(ccvTP)” (analysis result), “autonomic nerve function age” (analysis result), “autonomic nerve function age rank” (result of decision), “LF/HF rank” (result of decision), “LF/HF icon color” (result of decision), “LF/HF evaluation” (result of decision), “comprehensive decision rank” (result of decision“, “comprehensive decision icon color” (result of decision), “self-care advice” (result of decision), and “registration date and time” (processing date and time).

FIG. 12 is a view illustrating an example of the report of the fatigue level measurement result.

In the drawing, a fatigue level measurement result report 2300 includes a basic information and measurement information area 2301, an autonomic nerve function age area 2302, a heart rate variability area 2303, a sympathetic/parasympathetic nerve area 2304, and an evaluation area 2305.

The basic information and measurement information area 2301 is a display area for displaying a measurement environment of the measurer (subject) and an information related to the measurer (subject), wherein information at the time of measurement, measurement date, measurement time and so on are displayed in the relevant area.

### <Basic Information>

That is, the basic information and measurement information area 2301 shows the name, sexuality and age as the basic information of the subject, and measurement location and measurement time as the measurement information.

The autonomic nerve function age area 2302 is a display area in which a function age is displayed by comparing the autonomic nerve strength and strength deteriorated by aging with an average value of each age group, and in the area is further displayed the autonomic nerve function age at the time of measurement.

In the graph, the vertical axis represents autonomic nerve function (CCVTP), and the horizontal axis represents age.

The autonomic nerve function indicates the strength of the autonomic nerve (sympathetic nerve and parasympathetic nerve), and it can be seen that green line b drops with age by age average. A face mark H1 indicates, as function age, which age average the strength of the autonomic nerve corresponds to, based on the current measurement value. The mark moves upward as the autonomic nerve function is increased, and moves downward by tendency of fatigue and the like. A red line c is the low value of reference value of that age. It can be used as an index indicating that the function is deteriorated if the value falls below the line. The blue line a is the high value of the reference value of that age. If the value is higher, measurement noise is suspected, and re-measurement should be performed. If the value is the same after re-measurement, the value is considered to be correct, and it can be determined that the autonomic nerve function of the subject is extremely high.

### <Autonomic Nerve Function Age (Strength of Autonomic Nerve Function)>

In the autonomic nerve function age area 2302, the high value, the center value and the low value of CCVTP of each age from 20 to 70 are displayed in a graph as standard display.

In the drawing, (a) represents the high value, (b) represents the medium value, and (c) represents the low value.

On the graph, the value of CCVTP of the measurement result of the subject is plotted on the vertical axis, and the age of the subject is plotted on the horizontal axis.

A face mark is displayed on the plot, wherein if the measurement value is lower than the low value corresponding to the age of the subject, a yellow face mark is plotted, and in other cases, a blue face mark is plotted. The mark (d) represents the plotted face mark.

The superiority or inferiority with respect to the reference value is visually displayed in an intelligible manner by displaying yellow for caution and blue for normal.

The CCVTP of the measurement result and the relative function age are displayed below the graph.

The heart rate variability area 2303 is an area for displaying an average heart rate, wherein the average heart rate and fluctuation at the time of measurement is displayed in the area.

In other words, the measured average heart rate and the state of the heart rate variability (variability of heart beat intervals) are displayed in a graph. The portion where the waveform is dropped to the bottom indicates measurement failure (data deficiency).

### <Heart Rate Variability>

That is, the heart rate variability area 2303 displays the average heart rate based on the measurement result of the subject, and the reference value. In addition, the variability of heart rate is displayed in a line graph.

The sympathetic/parasympathetic nerve area 2304 indicates the balance between sympathetic nerve and parasympathetic nerve which constitute the autonomic nerve. It shows that the sympathetic nerve is dominant (when under tension or stress) as the face mark H2 is biased toward the right, and that the parasympathetic nerve is dominant (when relaxing) as the face mark is biased toward the left.

The sympathetic nerve and the parasympathetic nerve should be well-balanced, and in the ideal state, the sympathetic nerve is dominant during the active period and the parasympathetic nerve is dominant during the resting period.

### <Sympathetic and Parasympathetic Nerves (Autonomic Nerve Balance)>

The sympathetic/parasympathetic nerve area 2304 displays the LH/HF and the reference value based on the result of measurement of the subject.

The state of the measurement result of LH/HF is displayed by a supplementary comment.

A bar graph is displayed where the reference value (0.8 to 2.0) of the LH/HF is indicated by blue, a value smaller than 0.8 is indicated by yellow, and a value equal to or greater than 2.0 is indicated by red.

The LH/HF of the measurement result is plotted on the bar graph, and in doing so, a face mark whose color corresponds to the reference value is used.

The evaluation area 2305 is a display area displaying evaluation and comments based on the function age and balance of the autonomic nerve, and the area displays the result of decision of the autonomic nerve state during measurement, based on the function (strength) and balance. The description of the autonomic nerve state during measurement and advice for improving the autonomic nerve function (strength) and balance are displayed.

### <Autonomic Nerve Evaluation (Comprehensive Evaluation)>

The evaluation area 2305 displays a comprehensive evaluation and advice decided based on the autonomic nerve function age (autonomic nerve function strength) and the balance of the sympathetic/parasympathetic nerves.

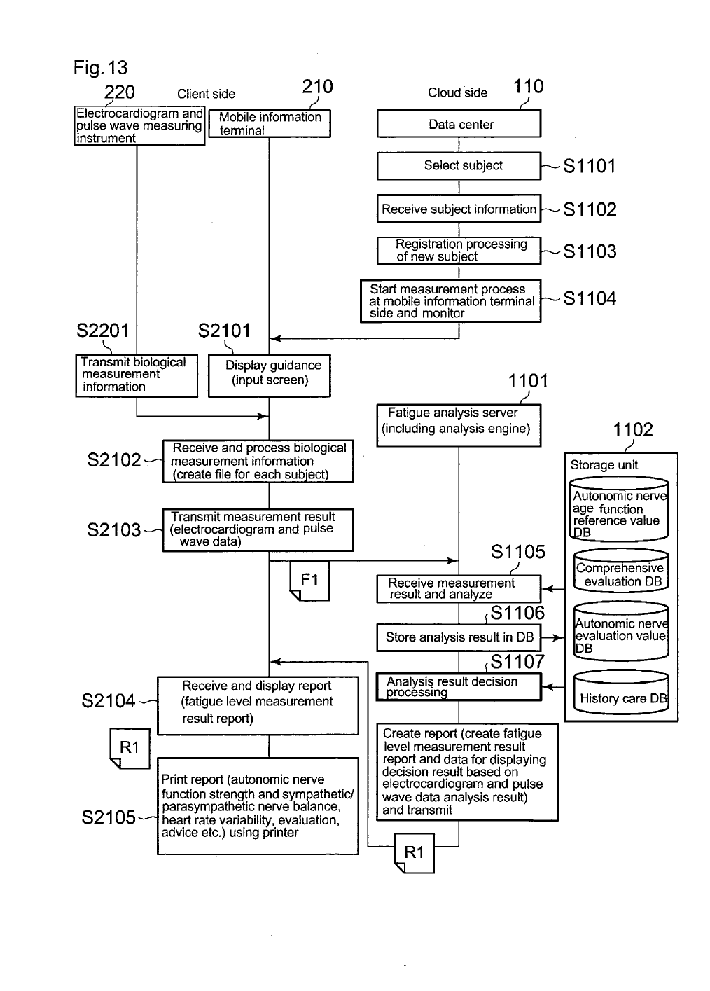

FIG. 13 is a view of the sequence of the electrocardiogram and pulse wave measuring instrument, the mobile information terminal, and the fatigue analysis server (including the analysis engine), and a process flow of the respective units.

In the drawing, at first in step S1101, the cloud-side data center 110 selects a subject, and in step S1102, it receives the subject information. Then, in step S1103, it performs a registration processing of a new subject, and in step S1104, it starts the measurement process at the mobile information terminal 210 side and monitors the same.

Further, in step S2201, the electrocardiogram and pulse wave measuring instrument 220 measures the biological measurement information (electrocardiogram and pulse wave data), and transmits the electrocardiogram and pulse wave data to the mobile information terminal 210.

In step S2101, the mobile information terminal 210 displays guidance according to the start of measurement processing from the data center 110. The subject at the mobile information terminal 210 side enters desired information according to the guidance. Further, in step S2102, the biological measurement information is received and processed. At this time, a file is created for each subject, and desirably, information is transmitted and received for each subject.

Then, in step S2103, the measurement result (electrocardiogram and pulse wave data) F1 is transmitted to the cloud-side fatigue analysis server 1101.

The fatigue analysis server 1101 receives the measurement result in step S1105, and analyzes the result. This analyzing process is performed by referring to the information in each DB of the storage unit 1102. Next, in step S1106, the analysis result is stored in the history care DB, and in step S1107, a report as described above (including the autonomic nerve function strength and sympathetic/parasympathetic nerve balance, heart rate variability, evaluation, and advice) R1 is created. When creating a report, data is created for displaying the report of the fatigue measurement result and the result of decision based on the electrocardiogram and pulse wave data analysis result. Then, the report R1 is transmitted to the mobile information terminal 210.

In step S2104, the mobile information terminal 210 receives the report R1, and displays the same on the display unit 2104 of the terminal. Further, in step S2105, it prints out the report R1 using the printer 230.

According to the present system, the electrocardiogram and pulse wave data measured by the electrocardiogram and pulse wave measuring instrument is analyzed using the cloud-side analysis server, so that the stress state can be comprehended by a numerical value based on the balance and strength of the autonomic nerve, and the relevant analysis data can be transmitted to the client terminal side to be visually displayed on the client terminal, by which a portable and convenient system can be configured where measurement can be performed easily and objectively by anyone in a short period of time.

According to the present embodiment, the electrocardiogram and pulse wave data measured by the electrocardiogram and pulse wave measuring instrument is analyzed using the cloud-side analysis server, so that the stress state can be comprehended by a numerical value based on the balance and strength of the autonomic nerve, and the relevant analysis data can be transmitted to the client terminal side to be visually displayed on the client terminal, by which a portable and convenient system can be configured where measurement can be performed easily and objectively by anyone in a short period of time.

The present invention is not restricted to the above-illustrated preferred embodiments, and can include various modifications. The above-illustrated embodiments are described in detail to help understand the present invention, and the present invention is not restricted to a structure including all the components illustrated above.

Further, a portion of the configuration of an embodiment can be replaced with the configuration of another embodiment, or the configuration of a certain embodiment can be added to the configuration of another embodiment.

A portion or whole of the above-illustrated configurations, functions, processing units, processing means and so on can be realized via hardware configuration such as by designing an integrated circuit.

Further, the configurations and functions illustrated above can be realized via software by the processor interpreting and executing programs realizing the respective functions. Information including the programs, tables and files for realizing the respective functions can be stored in a storage device such as a memory or a hard disk.

Only the control lines and information lines that are considered necessary for description are illustrated in the drawings, and not necessarily all the control lines and information lines required for production are illustrated. In actual application, it can be considered that almost all the components are mutually coupled.

### REFERENCE SIGNS LIST

10 Cloud-side device
110 Data center
1101 Analysis system (fatigue level analysis server)
11010 Analysis engine
110101 Electrocardiogram analysis unit
110102 Heart rate analysis unit
110103 Pulse wave analysis unit
110104 Autonomic nerve function analysis
11011 Biological data analysis unit
11012 DB retrieval and analysis result writing unit
11013 Commend adding unit
11014 Analysis report (fatigue level measurement result report) creation unit
11015 Analysis result decision unit
1102 Database (storage unit)
11021 History care DB
11022 Autonomic nerve age reference DB
11023 Autonomic nerve evaluation value DB
11024 Total evaluation value DB
1103 Data transmission and reception IF unit
20 Client-side device
210 Mobile information terminal
2101 Keyboard (input unit)
2102 Control unit (arithmetic processing unit)
2103 Data transmission and reception IF unit
2104 Display unit (output unit)
2105 Subject file creation unit
2106 Biological data reception IF unit
220 Biological measuring instrument
2201 Cardiograph and pulse wave data transmission IF unit
2202, 2203 Electrocardiogram and pulse measurement electrode
230 Output device (printer)

1.-2. (canceled)

3. A screening system for fatigue and stress analyzing and evaluating a fatigue and stress of a subject based on an electrocardiogram and pulse data of the subject and a reference value for each age based on an autonomic nerve state of each age, and generating an analysis report for enabling the subject to perform care corresponding to a condition of life based on an analysis and evaluation result of fatigue and stress, comprising:
a data transmission and reception interface unit receiving electrocardiogram and pulse data of a subject measured through a biological measuring instrument from a subject terminal of the subject, and transmitting an analysis report created based on the analysis and evaluation of the electrocardiogram and pulse data of the subject to the subject terminal;
an analysis unit analyzing fatigue and stress of the subject based on the electrocardiogram and pulse data and a heart rate variability by the biological measuring instrument;
a decision and evaluation unit for deciding and evaluating an analysis result by the analysis unit by comparison with the reference value;
a storage unit storing an analysis data of the subject by the analysis unit as history care information, and saving a reference value for each age based on the autonomic nerve state of each age as a master;
an analysis report creating means creating the analysis report; and
a commend addition unit for adding a comment related to a state of autonomic nerve balance according to the decision and evaluation result decided and evaluated by the decision and evaluation unit to the analysis report;
wherein the analysis unit comprises
an electrocardiogram and pulse data analysis unit analyzing a strength of the autonomic nerve (LF/HF) based on the electrocardiogram and pulse data of the subject, and comparing the strength of the autonomic nerve with the autonomic nerve age reference value by the reference value to analyze the degree of fatigue, a heart rate analysis unit analyzing an electrocardiogram variability from the electrocardiogram data of the subject, and an autonomic nerve function analysis unit analyzing the autonomic nerve balance (LF/HF), and comparing the analyzed balance with an autonomic nerve evaluation value (LF/HF evaluation value) of the reference value to analyze a stress tendency,
the decision and evaluation unit comprises
an autonomic nerve function comprehensive decision unit comprehensively deciding an autonomic nerve function based on the respective analysis results of the electrocardiogram and pulse data analysis unit, the autonomic nerve function analysis unit, and the heart rate analysis unit,
the storage unit comprising
a history care storage unit storing the analysis data as a history care information,
an autonomic nerve evaluation value storage unit storing the autonomic nerve evaluation value (LF/HF evaluation value),
an autonomic nerve age reference value storage unit storing the autonomic nerve age reference value, and
a comprehensive evaluation storage unit storing a comprehensive evaluation advice information corresponding to the result of the autonomic nerve function comprehensive decision performed by the autonomic nerve function comprehensive decision unit,
the analysis report creation unit comprising
an analysis report creation unit creating an analysis report including various information of advice and comments corresponding to the analysis result performed by the respective analysis units and a comprehensive evaluation in response to the decision result performed by the autonomic nerve function comprehensive decision unit,
the autonomic nerve evaluation value (LF/HF evaluation value) in the autonomic nerve evaluation value storage unit sets a number of the plurality of LF/HF ranks to M, and includes information of comments referring to a state for each of the number of the ranks,
the autonomic nerve age reference value in the autonomic nerve age reference value storage unit is set so that a number of the plurality of autonomic nerve function age ranks composed of the reference value of each age is N,
the comprehensive evaluation in the comprehensive evaluation storage unit sets a product of the LF/HF ranks M and the autonomic nerve function age ranks N as a number of the comprehensive evaluation ranks (M×N), and includes information of a number of advices corresponding to the product number, and
provides a result of the autonomic nerve function comprehensive decision performed by the autonomic nerve function comprehensive decision unit and a comprehensive evaluation advice corresponding to the plurality of comprehensive evaluation ranks by the analysis report, independent from the analysis result by each of the respective analysis units and the comment by the decision and evaluation unit.

4. The screening system for fatigue and stress according to claim 3, wherein
the biological measuring instrument is composed of an electrocardiogram and pulse measuring instrument capable of measuring electrocardiogram and pulse at the same time,
the autonomic nerve evaluation value (LF/HF evaluation value) of the storage unit and the autonomic nerve age reference value are set as master based on a measurement result having measured a reference value for each age based on the autonomic nerve state of each age, and
analysis and evaluation data by the analysis and evaluation unit, the subject data, and the measurement data measured by the biological measuring instrument are set so as not to be saved.

5. The screening system for fatigue and stress according to claim 3, wherein
the plurality of LF/HF ranks are composed of four classifications, which are “low value: 0.0 to 0.8”, “reference: 0.8 to 2.0”, “high value: 2.0 to 5.0”, and “extremely high value: 5.0 and greater”,
the plurality of autonomic nerve function age ranks are composed of three classifications, which are “smaller than low value”, “high value or higher”, and “standard value other than the former values”,
the comprehensive evaluation ranks are composed of 12 classifications, which are “four classifications of the LF/HF rank multiplied by three classifications of the autonomic nerve function age ranks”,
the comment is a comment of state for each of the four classifications of the LF/HF rank, and
the advice is an advice for each of the twelve classifications of the comprehensive evaluation ranks.
